(19) United States
(12) Patent Application Publication
(10) Pub. No.: US 2010/0004191 A1
(43) Pub. Date: Jan. 7, 2010

(54) Compositions of S-adenosyl-L-methionine.

(76) Inventor: Rolland F Hebert, Seattle, GA (US)

Correspondence Address: ROLLAND HEBERT, 427 BELLEVUE AVE E. SUITE 301, SEATTLE, WA, 98102, US

(21) Appl. No.: 12/166,312

(22) Filed: Jul. 1, 2008

(51) Int. Cl. A61K 31/7076; A61P 25/00

(52) U.S. Cl. 514/46

(57) ABSTRACT

Compositions of S-adenosyl-L-methionine with indole-3-propionic acid and methods to treat conditions associated with DNA hypomethylation are disclosed.

### CROSS-REFERENCES TO RELATED APPLICATION

This application claims the benefit of U.S. patent application Ser. No. 11/298,223 filed on Dec. 10, 2005 but now abandoned.

### BACKGROUND

1. Field of the Invention

The present invention relates to novel compositions of matter containing substantially optically pure diastereomers of S-adenosyl-L-methionine and defined non-racemic ratios of (S,S)—S-adenosyl-L-methionine to (R,S)—S-adenosyl-L-methionine and their pharmaceutically acceptable salts with indole-3-propionic acid. These compositions possess potent activity in treating various conditions associated with aging in mammals.

2. Technical Field

This patent relates to novel compositions of matter containing optically pure diastereomers of S-adenosyl-L-methionine (SAM-e), defined non-racemic ratios of (S,S)—S-adenosyl-L-methionine to (R,S)—S-adenosyl-L-methionine and their pharmaceutically acceptable salts and indole-3-propionic acid and to therapeutic uses of these new compositions.

### BACKGROUND OF THE INVENTION

The concept of aging refers to all the accumulated physiological changes of an organism over time and the aging aspects and speed differ in each individual case. Even in an individual, for example, a mammal such as a human, aging shows different aspects in each organ, so that individual organ or tissue oriented study on aging presents limitations. More particularly, aging changes cellular functions that result in changes of each organ and tissue and this in turn results in aging of the individual. For example, the damage of nerve cells of brain causes the decrease of cognition, the damage of subcutaneous fat cells causes the loss of the elasticity of skin, the loss of melanin generating capability of melanocytes of hair root causes white hair, etc.

The signs and symptoms of aging, for example in mammals including humans, can include, but are not limited to the following: decline in memory, aging of the skin, mild cognitive impairment, benign forgetfulness, muscle weakness and loss, osteoarthritis, osteoporosis, cardiovascular disease, increase incidence of cancer, increased insulin resistance and ultimately, increased fragility and other well know deleterious symptoms that accompany increased lifespan. While no scientific consensus yet exists to explain all the molecular changes that cause aging, some progress is being made in finding changes in genes that may be related to longevity and vitality.

Aging is also associated with global DNA hypomethylation. It has been observed that serial passaging of human skin fibroblasts to senescence results in global DNA hypomethylation. (Barciszewska M Z, Barciszewska A M, Rattan S I. mTLC-based detection of methylated cytosine: application to aging epigenetics. Biogerontology. 2007 December; 8(6):673-8).

One approach the present inventor has discovered is to influence or modify the aging process by modulating the methylation of DNA, RNA, proteins and other molecules using the novel compositions as disclosed herein. Using the compositions of the current invention to reverse or prevent the DNA hypomethylation that is a hallmark of the aging process, the inventor endeavors to modify the aging process and the symptoms associated with that process.

In order to decrease the deleterious effects of longevity, researchers are considering a number of approaches. One symptom of the aging process is mild cognitive impairment and this symptom can both be described objectively in animal models as well as modulated by various interventions, including pharmaceutical interventions such as the novel compositions as described herein.

Mild cognitive impairment (MCI) is an impairment in cognition, specifically memory performance, that is frequently associated with aging. The degree and type of impairment distinguishes MCI from dementia in that MCI patients exhibit deficits in secondary tests of memory, but perform normally on standard tests measuring other cognitive domains. Thus, MCI is defined as a clinical disorder that is distinct from early stages of dementia, particularly Alzheimer's type dementia, and can therefore be specifically targeted for treatment intervention.

The underlying causes of memory loss in MCI have not been determined, thus a strategy for treatment has not been easily identified. Although some investigators believe that most MCI patients have neuropathology that is characteristic of Alzheimer's disease, many patients diagnosed with MCI typically do not progress to Alzheimer's Disease, thereby suggesting that MCI has an underlying pathophysiology that is divergent from that of Alzheimer's despite other characteristics that may be shared.

### S-adenosyl-L-methionine

S-adenosyl-L-methionine is a naturally occurring substance that is present in all living organisms and has a number of very important biological functions. Among these functions are the following: methyl group donor in transmethylation reactions (it is the sole methyl group donor in such reactions-including methylation of DNA, proteins, hormones, catechol and indoleamines and phosphatidylethanolamine to phosphatidylcholine); it is a substrate of an enzyme lyase that converts S-adenosyl-L-methionine to the molecule methylthioadenosine and homoserine; it is an aminobutyric chain donor to tRNA; it is an aminoacidic chain donor in the biosynthesis of biotin; S-adenosyl-L-methionine, after decarboxylation, is the donor of aminopropyl groups for the biosynthesis of neuroregulatory polyamines spermidine and spermine. (Zappia et al (1979), Biomedical and Pharmacologcial roles of Adenosylmethionine and the Central Nervous System, page 1, Pergamon Press. NY.)

Many organic compounds exist in optically active forms, i.e., they have the ability to rotate the plane of plane-polarized light. In describing an optically active compound, the prefixes D and L or R and S are used to denote the absolute configuration of the molecule about its chiral center. The prefixes d and 1 or (+) and (−) are employed to designate the sign of rotation of plane-polarized light by the compound, with (−) or 1 meaning that the compound is levorotatory. A compound prefixed with (+) or d is dextrorotatory. For a given chemical structure, these compounds, called stereoisomers, are identical except that they are mirror images of one another. A specific stereoisomer may also be referred to as an enantiomer, and a mixture of such isomers is often called an enantiomeric mixture. A 50:50 mixture of enantiomers is referred to as a racemic mixture. A compound with more than one chiral center is a diastereomer. S-adenosyl-L-methionine is a diastereomer.

Stereochemical purity is of importance in the field of pharmaceuticals, where 12 of the 20 most prescribed drugs exhibit chirality. A case in point is provided by the L-form of the beta-adrenergic blocking agent, propranolol, which is known to be 100 times more potent than the D-enantiomer.

Furthermore, optical purity is important since certain isomers may actually be deleterious rather than simply inert. For example, it has been suggested that the D-enantiomer of thalidomide was a safe and effective sedative when prescribed for the control of morning sickness during pregnancy, and that the corresponding L-enantiomer was a potent teratogen.

S-adenosyl-L-methionine has been used clinically in the treatment of liver disease (Friedel H, Goa, K. L., and Benfield P., (1989), S-adenosyl-L-methionine: a review of its pharmacological properties and therapeutic potential in liver dysfunction and affective disorders in relation to its physiological role in cell metabolism. Drugs. 38, 389-416), arthritis (Di Padova C, (1987), S-adenosyl-L-methionine in the treatment of osteoarthritis: review of the clinical studies. Am J. Med. 83, (Suppl. 5), 6-65), and depression (Kagan, B, Sultzer D. L., Rosenlicht N and Gerner R. (1990), Oral S-adenosyl-L-methionine in depression: a randomized, double blind, placebo-controlled trial. Am. J. Psychiatry 147, 591-595.) Alzheimer's patients have reduced cerebral spinal fluid levels of S-adenosyl-L-methionine (Bottiglieri et al, (1990), Cerebrospinal fluid S-adenosyl-L-methionine in depression and dementia: effects of treatment with parenteral and oral S-adenosyl-L-methionine. J. Neurol. Neurosurg. Psychiatry 53, 1096-1098.)

In a preliminary study, S-adenosyl-L-methionine was able to produce cognitive improvement in patients with Alzheimer's disease. (Bottiglieri et al (1994), The clinical potential of admetionine (S-adenosyl-1-methioinine) in neurological disorders. Drugs 48, 137-152.) S-adenosyl-L-methionine brain levels in patients with Alzheimer's disease are also severely decreased. (Morrison et al, (1996), Brain S-adenosyl-L-methionine levels are severely decreased in Alzheimer's disease, Journal of Neurochemistry, 67, 1328-1331.) Patients with Parkinson's disease have also been shown to have significantly decreased blood levels of S-adenosyl-L-methionine. (Cheng et al, (1997), Levels of L-methionine S-adenosyltransferase activity in erythrocytes and concentrations of S-adenosyl-L-methionine and S-adenosylhomocysteine in whole blood of patients with Parkinson's disease. Experimental Neurology 145, 580-585.)

S-adenosyl-L-methionine levels in patients treated with the antineoplastic drug methotrexate are reduced. Neurotoxicity associated with this drug may be attenuated by co-administration of S-adenosyl-L-methionine. (Bottiglieri et al (1994), The Clinical Potential of Ademetionine (S-adenosyl-L-methionine) in neurological disorders, Drugs, 48 (2), 137-152.) Cerebral spinal fluid levels of S-adenosyl-L-methionine have been investigated in HIV AIDS dementia Complex/HIV encephalopathy and found to be significantly lower than in non-HIV infected patients. (Keating et al (1991), Evidence of brain methyltransferase inhibition and early brain involvement in HIV positive patients Lancet: 337:935-9.)

Oral S-adenosyl-L-methionine administration to patients with and without liver disease has resulted in increases in liver glutathione levels. (Vendemiale G et al, (1989), Effect of oral S-adenosyl-L-methionine on hepatic glutathione in patients with liver disease. Scand J Gastroenterol; 24: 407-15. Oral administration of S-adenosyl-L-methionine to patients suffering from intrahepatic cholestasis had improvements in both the pruritus as well as the biochemical markers of cholestasis. (Giudici et al, The use of admethionine (S-adenosyl-L-methionine) in the treatment of cholestatic liver disorders. Meta-analysis of clinical trials. In: Mato et al editors. Methionine Metabolism: Molecular Mechanism and Clinical Implications. Madrid: CSIC Press; 1992 pp 67-79.) Oral S-adenosyl-L-methionine administration to patients suffering from primary fibromyalgia resulted in significant improvement after a short term trial. (Tavoni et al, Evaluation of S-adenosylmethioine in Primary Fibromaylgia. The American Journal of Medicine, Vol 83 (suppl 5A), pp 107-110, 1987.) S-adenosyl-L-methionine has been used for the treatment of osteoarthritis as well. (Koenig B. A long-term (two years) clinical trial with S-adenosyl-L-methionine for the treatment of osteoarthritis. The American Journal of Medicine, Vol 83 (suppl 5A), Nov. 20, 1987 pp 89-94)

S-adenosyl-L-methionine is clinically useful in many apparently unrelated areas because of its important function in basic metabolic processes. One of its most striking clinical uses is in the treatment of alcoholic liver cirrhosis that, until now, remained medically untreatable. Mato et al demonstrated the ability of oral S-adenosyl-L-methionine in alcoholic liver cirrhosis to decrease the overall mortality and/or progression to liver transplant by 29% vs 12% as compared with a placebo treated group. (Mato et al (1999), S-adenosyl-L-methionine in alcohol liver cirrhosis: a randomized, placebo-controlled, double blind, multi-center clinical trial, Journal of Hepatology, 30, 1081-1089.)

S-adenosyl-L-methionine, incubated in vitro with human erythrocytes, penetrates the cell membrane and increases ATP within the cell thus restoring the cell shape. (Friedel et al, S-adenosyl-L-methionine: A review of its pharmacological properties and therapeutic potential in liver dysfunction and affective disorders in relation to its physiological role in cell metabolism, Drugs 38 (3):389-416, 1989).

S-adenosyl-L-methionine has been studied in patients suffering from migraines and found to be of benefit. (Friedel et al, S-adenosyl-L-methionine: A review of its pharmacological properties and therapeutic potential in liver dysfunction and affective disorders in relation to its physiological role in cell metabolism, Drugs 38 (3): 389-416, 1989).

Indole-3-propionic acid

Indole-3-propionic acid (3-(3-Indolyl) propionic acid) (IPA) is a natural compound found in plants and animals, including humans. It has been found in mammalian plasma and cerebrospinal fluid, including human plasma and cerebrospinal fluid. Indole-3-propionic acid is the most potent naturally occurring antioxidant known and is especially potent against hydroxyl radicals that are considered to be the most reactive and toxic of the oxygen derived free radicals. In contrast to other antioxidants known, indole-3-propionic acid does not convert to reactive metabolites and thus may be a superior antioxidant choice compared to other known antioxidants.

### PRIOR ART

S-adenosyl-L-methionine and its salts have only rarely been studied in animal models of cognitive impairment related to the aging process. In one study presented in a symposium, S-adenosyl-L-methionine was shown to improve cognition in both healthy aged as well as health young rats. Garcia P et al in “S-adenosylmetionine: A drug for the brain?”, IV th Workshop on Methionine Metabolism: Molecular Mechanisms and Clinical Implications”, Symposium held on Mar. 1-5, Granada, Spain, 1998. Indole-3-propionic acid as a therapeutic intervention for mild cognitive impairment has not been reported in the literature.

### SUMMARY OF THE INVENTION

Briefly stated, the present invention discloses compositions of S-adenosyl-L-methionine and its pharmaceutically acceptable salts and substantially optically pure diastereomeric forms of S-adenosyl-L-methionine, defined non-racemic ratios of (S,S)—S-adenosyl-L-methionine to (R,S)—S-adenosyl-L-methionine and their salts with indole-3-propionic acid. The compositions of this present invention have utility in increasing blood, RNA methylation and DNA methylation levels and other tissue or fluid levels of S-adenosyl-L-methionine, as well as treating conditions in warm blooded animals associated with the aging process or low RNA methylation, DNA methylation, protein methylation, blood or other tissue or fluid levels of S-adenosyl-L-methionine.

Thus in one embodiment, a composition of S-adenosyl-L-methionine and its pharmaceutically acceptable salts and a substantially optically pure diastereomeric form of S-adenosyl-L-methionine salt or defined non-racemic ratios of (S,S)—S-adenosyl-L-methionine to (R,S)—S-adenosyl-L-methionine and their salts with indole-3-propionic acid is administered to a warm-blooded animal in need thereof to increase tissue, cellular RNA methylation, DNA methylation, protein methylation and blood S-adenosyl-L-methionine levels.

In yet a further embodiment, a composition of S-adenosyl-L-methionine and its pharmaceutically acceptable salts and a substantially optically pure diastereomeric form of S-adenosyl-L-methionine salt or defined non-racemic ratios of (S,S)—S-adenosyl-L-methionine to (R,S)—S-adenosyl-L-methionine and their salts with indole-3-propionic acid of the present invention is administered to a warm blooded animal to treat the following conditions: memory disturbances, memory loss, mild cognitive impairment associated with the aging process.

In yet a further embodiment, a composition of S-adenosyl-L-methionine and its pharmaceutically acceptable salts and a substantially optically pure diastereomeric form of S-adenosyl-L-methionine salt or defined non-racemic ratios of (S,S)—S-adenosyl-L-methionine to (R,S)—S-adenosyl-L-methionine and their salts with indole-3-propionic acid of the present invention is administered to a warm blooded animal to increase DNA methylation, RNA methylation, protein methylation.

### DETAILED DESCRIPTION OF THE INVENTION

As mentioned above, this invention is generally directed to compositions of S-adenosyl-L-methionine and its pharmaceutically acceptable salts and a substantially optically pure diastereomeric form of S-adenosyl-L-methionine salts and to defined non-racemic ratios of (S,S)—S-adenosyl-L-methionine to (R,S)—S-adenosyl-L-methionine and their salts with indole-3-propionic acid. Such a composition of S-adenosyl-L-methionine and its pharmaceutically acceptable salts and a substantially optically pure diastereomeric form of S-adenosyl-L-methionine salt or defined non-racemic ratios of (S,S)—S-adenosyl-L-methionine to (R,S)—S-adenosyl-L-methionine and their salts with indole-3-propionic acid of the present invention is administered to a warm-blooded animal in need thereof to treat a condition associated with, DNA hypomethylation, RNA hypomethylation, protein hypomethylation, and low tissue and blood levels of S-adenosyl-L-methionine.

As used herein, the term “conditions” includes aging, diseases, injuries, disorders, indications and/or afflictions that are associated with decreased levels of blood and tissue S-adenosyl-L-methionine as well as decreased RNA methylation, DNA methylation, protein methylation. The term “treat” or “treatment” means that the symptoms associated with one or more conditions associated with low levels of S-adenosyl-L-methionine or DNA hypomethylation, RNA hypomethylation or protein hypomethylation are alleviated or reduced in severity.

The term “substantially optically pure as used herein, means that the composition contains greater than about 82.0% of the (S,S)—S-adenosyl-L-methionine diastereomer by weight in relation to the (R,S) diastereomer of S-adenosyl-L-methionine, preferably greater than about 90% of the (S,S)—S-adenosyl-L-methionine by weight, and more preferably greater than about 96.00% of (S,S)—S-adenosyl-L-methionine by weight, based upon the total weight of S-adenosyl-L-methionine.

The substantially optically pure diastereomeric forms of S-adenosyl-L-methionine salts or defined non-racemic ratios of (S,S)—S-adenosyl-L-methionine to (R,S)—S-adenosyl-L-methionine and their salts as well as indole-3-propionic acid envisioned in this patent may be used to treat a variety of conditions associated with lowered levels of S-adenosyl-L-methionine.

Due to its ubiquitous distribution in mammalian tissue, S-adenosyl-L-methionine is associated with a variety of conditions: aging, aging of the skin, Alzheimer's disease, rheumatoid arthritis, osteoarthritis, both as an anti-inflammatory as well as to promote new cartilage formation, cancer, conditions of hypomethylation, mitochondrial diseases, hypomethylation of DNA and RNA and proteins, HIV/AIDS, anxiety, attention deficit disorder and ADHD, sleep regulation, organ preservation for transplant industry, dyslipidemias, excess sebum production, migraines, bile dysfunction caused by pregnancy and use of contraceptive medications, depression, acute and chronic liver disease, cirrhosis of the liver, ischemic reperfusion injury, Parkinson's disease, memory disturbances, memory loss, pancreatitis, intrahepatic cholestasis, inflammation, pain, side effects of administration of chemotherapy, liver disease associated with administration of total parenteral nutrition, liver dysfunction, low tissue levels of glutathione, administration of neuroleptic drugs, administration of cyclosporin A, asthma, and alcohol-withdrawal.

Accordingly, compositions of S-adenosyl-L-methionine and its pharmaceutically acceptable salts and optically pure diastereomers of S-adenosyl-L-methionine or defined non-racemic ratios of (S,S)—S-adenosyl-L-methionine to (R,S)—S-adenosyl-L-methionine and their salts with indole-3-propionic acid of the present invention are effective in treating conditions associated with aging due to their ability to increase S-adenosyl-L-methionine levels or to increase RNA methylation, DNA methylation, or protein methylation. To this end, compositions of the present invention may be used for pharmaceutical, prophylactic and/or cosmetic purposes, and are administered to a warm-blooded animal in an effective amount to achieve a desired result.

In the case of pharmaceutical administration, an effective amount of the composition is a quantity sufficient to treat the symptoms of a condition and/or the underlying condition itself. An effective amount of the composition in the context of prophylactic administration means an amount sufficient to avoid or delay the onset of a condition and/or its symptoms. Lastly, an effective amount with regard to cosmetic administration is an amount sufficient to achieve the desired cosmetic result.

In the current medical context, an effective amount of S-adenosyl-L-methionine and its pharmaceutically acceptable salts and a substantially optically pure diastereomeric form of S-adenosyl-L-methionine salts or a non-racemic mixture of (S,S)—S-adenosyl-L-methionine and (R,S)—S-adenosyl-L-methionine and their salts would be in the range of from 200 mg per day to 5 grams per day or greater depending upon the specific condition of the patient to be treated or prevented. It is well known in the medical art how one arrives at the appropriate dosage. In addition, it is well known in the art how one arrives at the determination of both blood and tissue levels of S-adenosyl-L-methionine as well as indole-3-propionic acid envisioned by this present patent application since NMR, HPLC as well as capillary electrophoresis methods are well known in the art to determine S-adenosyl-L-methionine.

In the current medical context, an effective amount of indole-3-propionic acid would be in the range of from about 200 mg per day to about 3 grams or more per day depending upon the specific condition of the patient to be treated. It is well known in the medical art how one arrives at the appropriate dosage.

In the current medical context, an effective amount of a composition of S-adenosyl-L-methionine and its pharmaceutically acceptable salts and a substantially optically pure diastereomeric form of S-adenosyl-L-methionine salts or a non-racemic mixture of (S,S)—S-adenosyl-L-methionine and (R,S)—S-adenosyl-L-methionine and their pharmaceutically acceptable salts with indole-3-propionic acid combined, for example, in a pill or capsule form would be in the range of between 100 mg of S-adenosyl-L-methionine and its pharmaceutically acceptable salts and a substantially optically pure diastereomeric form of S-adenosyl-L-methionine salts or a non-racemic mixture of (S,S)—S-adenosyl-L-methionine and (R,S)—S-adenosyl-L-methionine and their pharmaceutically acceptable salts and 100 mg of indole-3-propionic acid and 400 mg of S-adenosyl-L-methionine and its pharmaceutically acceptable salts and a substantially optically pure diastereomeric form of S-adenosyl-L-methionine salts or a non-racemic mixture of (S,S)—S-adenosyl-L-methionine and (R,S)—S-adenosyl-L-methionine and their pharmaceutically acceptable salts and 300 mg of indole-3-propionic acid. The percentages by weight of each of the constituents of the capsule or tablets or pills may vary but the most preferred combination of constituents is 400 mg of S-adenosyl-L-methionine and its pharmaceutically acceptable salts and a substantially optically pure diastereomeric form of S-adenosyl-L-methionine salts or a non-racemic mixture of (S,S)—S-adenosyl-L-methionine and (R,S)—S-adenosyl-L-methionine and their pharmaceutically acceptable salts and 200 mg of indole-3-propionic acid. Pills, tablets or capsules of the present invention may be given once, twice, or more times per day as deemed necessary to achieve the desired clinical effect

### EMBODIMENTS

It is an object of the present invention to provide compositions of S-adenosyl-L-methionine containing substantially optically pure biologically active (S,S)S-adenosyl-L-methionine or a defined non-racemic ratio of (S,S)—S-adenosyl-L-methionine to (R,S)—S-adenosyl-L-methionine and indole-3-propionic acid.

It is an object of the present invention to provide methods of treatment for memory deficits in a warm blooded animal in need thereof by administering the compositions of S-adenosyl-L-methionine containing substantially optically pure biologically active (S,S) S-adenosyl-L-methionine or a defined non-racemic ratio of (S,S)—S-adenosyl-L-methionine to (R,S)—S-adenosyl-L-methionine and indole-3-propionic acid.

It is also an object of the present invention to provide methods of treatment for conditions associated with DNA hypomethylation including aging and mild cognitive impairment, RNA hypomethylation, protein hypomethylation in a warm blooded animal in need thereof by administering the compositions of S-adenosyl-L-methionine containing substantially optically pure biologically active (S,S)S-adenosyl-L-methionine or a defined non-racemic ratio of (S,S)—S-adenosyl-L-methionine to (R,S)—S-adenosyl-L-methionine and indole-3-propionic acid.

In a preferred embodiment, substantially optically pure diastereomeric forms of S-adenosyl-L-methionine salts or a non-racemic mixture of (S,S)—S-adenosyl-L-methionine and (R,S)—S-adenosyl-L-methionine and their salts and indole-3-propionic of this current patent application are administered to a warm-blooded animal as a pharmaceutical, prophylactic or cosmetic composition containing at least one substantially optically pure diastereomeric form of S-adenosyl-L-methionine salt or a non-racemic mixture of (S,S)—S-adenosyl-L-methionine and (R,S)—S-adenosyl-L-methionine and their salts in combination with at least one pharmaceutically, prophylactically or cosmetically acceptable carrier or diluent. Administration may be accomplished by systemic or topical application, with the preferred mode dependent upon the type and location of the conditions to be treated. Frequency of administration may vary, and is typically accomplished by daily administration. In a preferred embodiment a composition useful for the treatment of conditions associated with RNA or DNA hypomethylation comprising substantially optically pure (S,S)—S-adenosyl-L-methionine or a defined non-racemic ratio of (S,S)S-adenosyl-L-methionine to (R,S)S-adenosyl-L-methionine and their pharmaceutically acceptable salts and indole-3-propionic acid are provided.

In another embodiment, a pharmaceutical composition of the non-racemic ratio of (S,S)—S-adenosyl-L-methionine to (R,S)—S-adenosyl-L-methionine (and their salts) is preferably from about 80.01% to about 100% of (S,S)—S-adenosyl-L-methionine to about 19.99% to about 0.0% by weight of (R,S)—S-adenosyl-L-methionine and indole-3-propionic acid of this current patent application are in the doses mentioned above.

In yet another embodiment, a pharmaceutical composition of the non-racemic ratio of (S,S)—S-adenosyl-L-methionine to (R,S)—S-adenosyl-L-methionine is more preferably from about 82% to about 96.9% of (S,S)—S-adenosyl-L-methionine to about 18% to about 3.1% by weight of (R,S)—S-adenosyl-L-methionine.

Systemic administration may be achieved, for example, by injection (e.g., intramuscular, intravenous, subcutaneous or intradermal) or oral delivery of the composition to the warm-blooded animal. Suitable carriers and diluents for injection are known to those skilled in the art, and generally are in the form of an aqueous solution containing appropriate buffers and preservatives. Oral delivery is generally accomplished by formulating the composition in a liquid or solid form, such as a tablet or capsule, by known formulation techniques.

Topical administration may be accomplished, for example, by formulating the composition as solution, cream, gel, ointment, powder, paste, gum or lozenge using techniques known to those skilled in the formulation field. As used herein, topical administration includes delivery of the composition to mucosal tissue of the mouth, nose and throat by, for example, spray or mist application, as well as to the vagina and rectum by, for example, suppository application.

The inventor discovers that by combining S-adenosyl-L-methionine and/or its optically pure diastereomeric forms of S-adenosyl-L-methionine salts or defined non-racemic ratios of (S,S)—S-adenosyl-L-methionine to (R,S)—S-adenosyl-L-methionine and their salts with indole-3-proponic acid, a synergistic advantageous effect on cognition in an animal model and DNA methylation in an in vitro model of cell aging.

The following examples show the unexpected synergist effects the novel compositions of the present invention have on both an in vivo as well as in vitro model of some aspects of the aging process.

The following examples also show how substantially optically pure diastereomeric forms of S-adenosyl-L-methionine salts or defined non-racemic ratios of (S,S)—S-adenosyl-L-methionine to (R,S)—S-adenosyl-L-methionine and their salts and indole-3-propionic acid of this present invention may be used. These examples are given to illustrate the present invention, but not by way of limitation. Accordingly, the scope of this invention should be determined not by the embodiments illustrated, but rather by the appended claims and their legal equivalents.

### Example 1

Animal model of cognitive impairment associated with the aging process

### Experimental Protocol

To investigate the influence of S-adenosyl-L-methionine: indole-3-propionic acid composition on learning and memory abilities in old rats, two groups (30 each) aged 12 months old, are injected subcutaneously with S-adenosyl-L-methionine: indole-3-propionic acid composition (consisting of 20 mg/kg/day of 90% (S,S)S-adenosyl-L-methionine 1,4 butanedisulfonate and 20/mg/kg/day of indole-3-propionic acid) during 10 months (treated group) or vehicle (control group).

To investigate the influence of S-adenosyl-L-methionine: indole-3-propionic acid composition in learning and memory abilities in young rats, two groups (25 each) aged 3 months old, are injected with S-adenosyl-L-methionine: indole-3-propionic acid composition consisting of 20 mg/kg/day of 90% (S,S)S-adenosyl-L-methionine 1,4 butanedisulfonate and 20/mg/kg/day of indole-3-propionic acid during 3 months (treated group) or vehicle (control group). Animals are housed individually at constant temperature (22±3° C.) under a fixed 12 hours light-dark cycle, with free access to food and water.

### To Investigate Learning and Memory:

An eight arms radial maze is used to test spatial working memory. A central octagonal chamber is equipped with eight radial arms. Arms can be reached only from the central chamber through an entrance with a gate. After treatment with S-adenosyl-L-methionine: indole-3-propionic acid composition or vehicle, rats are trained in the maze during a period of two weeks. A food restriction schedule is used during these two weeks, as food is the reward in the maze performance.

During the first days of training, rats are allowed to stay in the maze for 10 min, and food pellets are scattered along the arms. On successive days, food is restricted to the end of the arms, until on day 5, it is only available at the end of each arm. Food is located in a food cup so the animals cannot see it. To mask any odor clues, food pellets are scattered outside the walls of the maze. Rats are deprived of food 48 hours before the final test. The food-deprived animals are placed at the center of the maze and are allowed to adapt for 10 seconds with the gates closed. After the habituation period, the gates are lifted and rats can move freely anywhere in the maze. The session is terminated after a rat consumes the food in all the arms, or after a period of 10 min. In the analysis of the test the number of correct choices is evaluated, considering a correct choice the visit to an arm previously unvisited. The animals have to learn to visit each arm once, and to remember the location they have visited, as no new food is to be provided in arms already visited. Data between treated an control animals in each group is compared using the Man-Whitney test, setting the statistical significance at p<0.05.

Indole-3-propionic acid at >99% purity is available from Acros Organics/Global Chemicals Thermo Fisher Scientific, 500 The American Road, Morris Plains, N.J. 07950.

Morris R (1984). “Developments of a water-maze procedure for studying spatial learning in the rat”. J Neurosci Methods 11 (1): 47-60

### Example 2

### In Vitro Study of Antiaging Properties of S-Adneosyl-L-Methionine

### Indole-3-Propionic Acid Composition

It is known that serially passaged human skin fibroblast cells lose DNA methylation. The mechanism for this global DNA loss is unknown but this DNA hypomethylation may be used as a biomarker for cellular senescence. (Barciszewska M Z, Barciszewska A M, Rattan S I. mTLC-based detection of methylated cytosine: application to aging epigenetics. Biogerontology. 2007 December; 8(6):673-8).

To assess the effect of the composition of the present invention on both DNA hypomethylation as well as the effect of the composition on the number of population doublings in this in vitro assay, 100 micromolar of 90% (S,S)S-adenosyl-L-methionine 1,4 butanedisulfonate and 10 micromolar indole-3-propionic acid are added to the culture medium. In long-term treatment, cells are maintained through their replicative lifespan in the presence of 100 micromolar of 90% (S,S)S-adenosyl-L-methionine 1,4 butanedisulfonate and 10 micromolar indole-3-propionic acid added to the culture medium.

The passaging protocol is taken from Cell Stress Chaperones. 2004 March; 9(1): 49-57. Mild heat stress stimulates 20S proteasome and its 11S activator in human fibroblasts undergoing aging in vitro. Rasmus Beedholm, Brian F. C. Clark, and Suresh I. S. Rattan and is incorporated herein by reference in its entirety as are all references in this patent application.

Global DNA methylation status is determined at start and at end of experiment. The DNA methylation determination is carried out according to the method described by Barciszewska M Z, Barciszewska A M, Rattan S I. mTLC-based detection of methylated cytosine: application to aging epigenetics. Biogerontology. 2007 December; 8(6):673-8).

I claim:

1. A composition useful for the treatment of conditions associated with RNA or DNA hypomethylation including aging and mild cognitive impairment comprising substantially optically pure (S,S)S-adenosyl-L-methionine or a defined non-racemic ratio of (S,S)S-adenosyl-L-methionine to (R,S)S-adenosyl-L-methionine and their pharmaceutically acceptable salts and indole-3-propionic acid.

2. The composition of claim 1 in which the defined non-racemic ratio of (S,S)S-adenosyl-L-methionine:(R,S)S-adenosyl-L-methionine is from about 82% to 96.9%:18% to 3.1% by weight.
